(19) United States
(12) Patent Application Publication
(10) Pub. No.: US 2016/0030239 A1
(43) Pub. Date: Feb. 4, 2016

(54) PUPIL EXPANDER

(71) Applicant: FRONTIER VISION CO., LTD., Hyogo (JP)

(72) Inventors: Junsuke Akura, Wakayama (JP); Kiran Pkharel, Hyogo (JP)

(73) Assignee: Frontier Vision Co., LTD., Hyogo (JP)

(21) Appl. No.: 14/780,726

(22) PCT Filed: Mar. 10, 2014
(86) PCT No.: PCT/JP2014/056153

§ 371 Date: Sep. 28, 2015

(30) Foreign Application Priority Data
Mar. 29, 2013 (JP) 2013-071303

(51) Int. Cl. A61F 9/007; A61B 17/02

(52) U.S. Cl. CPC A61F 9/007; A61B 17/0231; A61F 9/00736; A61B 2017/00946

(57) ABSTRACT

The purpose of the present invention is to provide a pupil expander capable of being safely arranged without damaging an iris and capable of maintaining a sufficiently expanded state of a pupil during ophthalmologic surgery such as cataract surgery. This pupil expander 1 is used for maintaining a pupil P in an expanded state during cataract surgery and include: four arm sections 10 serially arranged so as to form a circular shape; and coupling sections 20 each made of a plastic material and configured to connect end sections of adjacent arm sections 10. The end sections of adjacent arm sections 10 are connected to each other by the coupling section 20 made of a plastic material and, as a result, the overall shape of the pupil expander 1 changes inside an eye on the basis of deformation of the coupling section 20 and the overall shape of the pupil expander 1 is maintained after the deformation. As a result, the pupil expander can be safely arranged without damaging an iris I and a sufficiently expanded state of the pupil P can be maintained.

### TECHNICAL FIELD

The present invention relates to a pupil expander to be used to maintain an expanded state of a pupil during ophthalmologic surgery such as, e.g., cataract surgery.

### BACKGROUND TECHNIQUE

Normally, a human eye has a function of adjusting an amount of light entering through a pupil positioned in a center of an iris. An iris is a ring-shaped tela positioned behind a cornea and forward of a crystalline lens, and has a central opening forming a pupil. An iris is structured by stretchable rough connective tissues and muscles for expanding and contracting a pupil. In a bright place, the tela of the iris expands centripetally to reduce the pupil (contract the pupil) so that the amount of light entering into the eye is reduced. In a dark place, the tela of the iris shrinks toward its root (peripheral portion of the pupil) to enlarge the pupil (expand the pupil) so that the amount of light entering into the eye is increased.

In the meantime, as one of diseases occurring in a human eye, there is a disorder called cataract which causes clouding of the crystalline lens with aging, resulting in reduced visual acuity. In a current cataract surgery, it is a mainstream to apply ultrasound crystalline lens emulsification suction surgery and intraocular lens insertion surgery. In this surgery, a circular incision of about 5 to 6 mm is formed in the center of the anterior capsule of the lens capsule. Then, the contents of the opacified crystalline lens are removed by suction via the incision, and an intraocular lens is inserted into the lens capsule through the incision.

In order to complete surgery of a crystalline lens, a vitreous body, or a retina inside an eye ball positioned behind an iris, such as, e.g., cataract surgery or vitreoretinal surgery, it is required to maintain the pupil in a sufficiently expanded (mydriasis) state (about 5 to 6 mm) during the surgery. However, in the case of an eye in which inflammation was present in the iris in the past, an eye in which eye drops of miotic agent were used for a long period of time for glaucoma, an eye in which pseudoexfoliation materials are adhered to an iris, or an eye in which senile pupillary constriction is strong, there is a case in which the pupil cannot be brought into a sufficiently expanded state even if a mydriatic agent is used before the surgery.

For an eye that the pupil cannot be brought into a fully expanded state as mentioned above, an iris retractor having a tip end section formed into a hook shape has been conventionally used (see, for example, the following Patent Document 1). The following explanation will be made by exemplifying a case using four iris retractors. When arranging these iris retractors, incisions are formed at four portions of the cornea and retractors are inserted into respective incisions. Then, the tip end sections of iris retractors are hooked to portions of the pupillary edge to pull the pupillary edge of the iris radially outward. In this state, using a silicon stopper, each of the iris retractor is fixed to the cornea to thereby maintain the state in which the diameter of the pupil is expanded to a sufficient size. Further, when removing the iris retractors, after loosening the fixing of the silicon stopper, the iris retractors are removed respectively. Such installations and removals of the iris retractors should be carefully performed so as not to damage the iris and/or crystalline lens, which requires considerable effort and time. Especially, since the hooking portion for hooking the pupillary edge is made by a thin resin wire, there has been a problem that the pupillary edge of the iris is torn off when the pupillary edge of the iris is pulled by the iris retractor, resulting in deformation of the pupil after the surgery.

On the other hand, in recent years, as a device for expanding a pupil more simply and in a shorter time than the iris retractors mentioned above, a pupil expander, such as, e.g., Malyugin Ring, The OASIS Iris Expander, Morcher Pupil Dilator, is known (see, for example, the following Patent Documents 2 to 4). These pupil expanders are constituted by plastic high in shape memory property, and formed into a square shape in a natural state, or an approximately ring-shape with one opened section. The pupil expander of this kind is stored in a dedicated injector in an elongate folded manner, and introduced into an eye through a small incision of about 2.2 to 3.2 mm to be fitted to the pupillary edge of the iris. The pupil expander expands the pupil radially outward with the pupillary edge of the iris hooked by iris engaging portions, which are formed at 4 to 5 portions or formed along approximately the entire circumference, from the inner side.

### PRIOR ART DOCUMENT

### Patent Document

Patent Document 1: Japanese Unexamined Laid-open Application Publication No. H7-194643

Patent Document 2: Japanese Translation of PCT International Application Publication No. 2010-521229

Patent Document 3: Japanese Translation of PCT International Application Publication No. 2002-531170

Patent Document 4: Japanese Translation of PCT International Application Publication No. H9-505753

### SUMMARY OF THE INVENTION

### Problems to be Solved by the Invention

However, the conventional pupil expander is made of a material high in shape memory property. Therefore, when released in an eye from the injector, the pupil expander returns to its original inherent shape (the shape in the natural state) inside the eye. Therefore, it expands into a square shape or a circular shape having a diameter of about 5 to 7 mm on the iris in the eye. For this reason, after hooking the iris engaging portion of the pupil expander with the pupillary edge using a medical device such as, e.g., a Sinskey hook, when hooking the adjacent or opposite side pupillary edge with another iris engaging portion, it is required to eccentrically move the iris engaging portion with the pupil wide opened. In view of this, since the difficulty of the work is not fully resolved, when arranging the pupil expander, there was a possibility that the iris tissue is damaged by the contact to the corneal endothelium important for maintaining the transparency of the cornea or the excessive stretching of the iris tissue.

Further, these pupil expanders can be placed in an eye in a moderate degree pupil expanded state. However, it is very difficult to place it to a pupil poor in the pupil expanded state or small in size, and therefore the pupil must be eccentrically expanded largely, which easily causes damages to the iris tissue.

Furthermore, in a conventional pupil expander, such as a Malyugin Ring, in order to compensate for the disadvantage that the expander returns to its original shape when inserted into an eye, the pupil expander is hooked to the pupillary edge while releasing the expander little by little in a state in which the injector is inserted in the eye. However, hooking the pupil expander to the pupillary edge of the iris is not easy itself, and therefore it is not easy for everyone to perform this operation. It becomes a very difficult task especially in cases where the pupil diameter is less than 3 mm. As for the removal, although the Malyugin Ring, etc., can be accommodated into the injector within the eye, there is a possibility that the iris may be damaged by the work.

The present invention was made in view of the aforementioned problems, and aims to provide a pupil expander capable of being arranged safely without damaging an iris during ophthalmologic surgery such as, e.g., cataract surgery, and also capable of maintaining a sufficiently expanded state of a pupil.

### Means for Solving the Problems

In order to attain the aforementioned object, the present invention is characterized in that a pupil expander to be used to maintain an expanded state of a pupil during ophthalmologic surgery such as, e.g., cataract surgery, includes four or more arm sections serially arranged in a manner as to form a circular shape, and coupling sections each made of a plastic material and configured to connect end sections of the arm sections arranged adjacently.

With this, since end sections of adjacent arm sections are connected via the coupling section made of a plastic material, the overall shape of the pupil expander can be deformed in an eye on the basis of deformation of the coupling section, and the overall shape of the pupil expander can be maintained after the deformation. For this reason, the pupil expander can be arranged safely without damaging an iris, which enables to maintain a sufficiently expanded state of a pupil.

Further, it is preferable that the coupling sections are formed into an overall circular shape in a state in which adjacent coupling sections are connected to each other, and the four or more arm sections are arranged at predetermined intervals in a circumferential direction of the coupling section. With this, the pupil expander can be safely arranged without damaging an iris and it becomes possible to maintain a sufficiently expanded state of a pupil.

Further, it is preferable that the coupling sections are each formed into a plate shape having a plane extending in an axial direction of a circular shape formed by the four or more arm sections. With this, the coupling section can be easily bent in a planar direction of the pupil expander, resulting in easy movements of each arm section in the planner direction of the pupil expander, which enables easy deformation of the overall shape of the pupil expander in the planner direction. Further, the coupling section is wide in the axial direction, so that the coupling section comes into contact with the pupillary edge of the iris, which enables to simply and assuredly maintain the expanded state of the pupil.

Further, it is preferable that the coupling sections are each formed by a plurality of coupling pieces provided in a manner as to be arranged in parallel to an axial direction of a circular shape formed by the four or more arm sections. With this, the arm section is supported at plurality portions along the axial direction, which enables to move each arm section in a planner direction in a stable manner.

Further, it is preferable that the coupling sections are each provided with an iris engagement assisting section for engaging a pupillary edge of an iris. With this, since the pupillary edge of the iris is engaged with the iris engagement assisting section, the pupil expander can be arranged more safely, and the expanded state of the pupil can be maintained more assuredly.

Further, it is preferable that the coupling sections are each provided with a device insertion hole for inserting a medical device for ophthalmologic surgery. With this, by inserting a medical device for ophthalmologic surgery in the device insertion hole, it becomes possible to apply a force radially inward or radially outward of the coupling section, which enables easy and assured deformations of the overall shape of the pupil expander within an eye.

Further, it is preferable that the arm section is curved in a manner as to expand radially outward. With this, since each arm section is arranged near an iris edge or outside the iris edge, the surgical field of the pupil section can be widened, enabling more easy surgery.

### Effects of the Invention

According to the present invention, since end sections of adjacent arm sections are connected by the coupling section made of a plastic material, the overall shape of the pupil expander can be deformed in an eye on the basis of the coupling section. This enables safe arrangement without damaging an iris, which in turn can maintain a sufficiently expanded state of the pupil. For this reason, any operators can arrange and remove the pupil expander at ease, which in turn can reduce the effort and time for surgery.

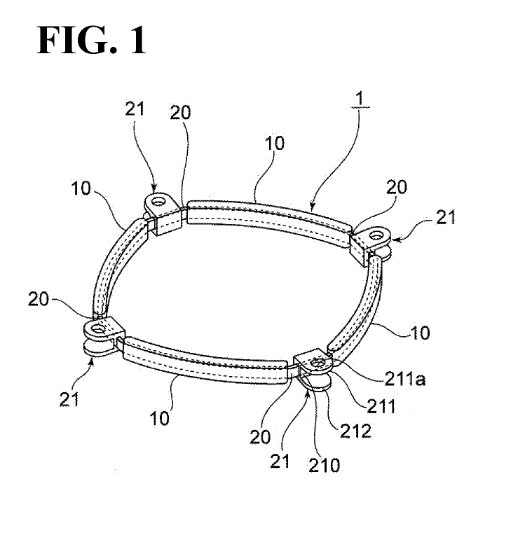

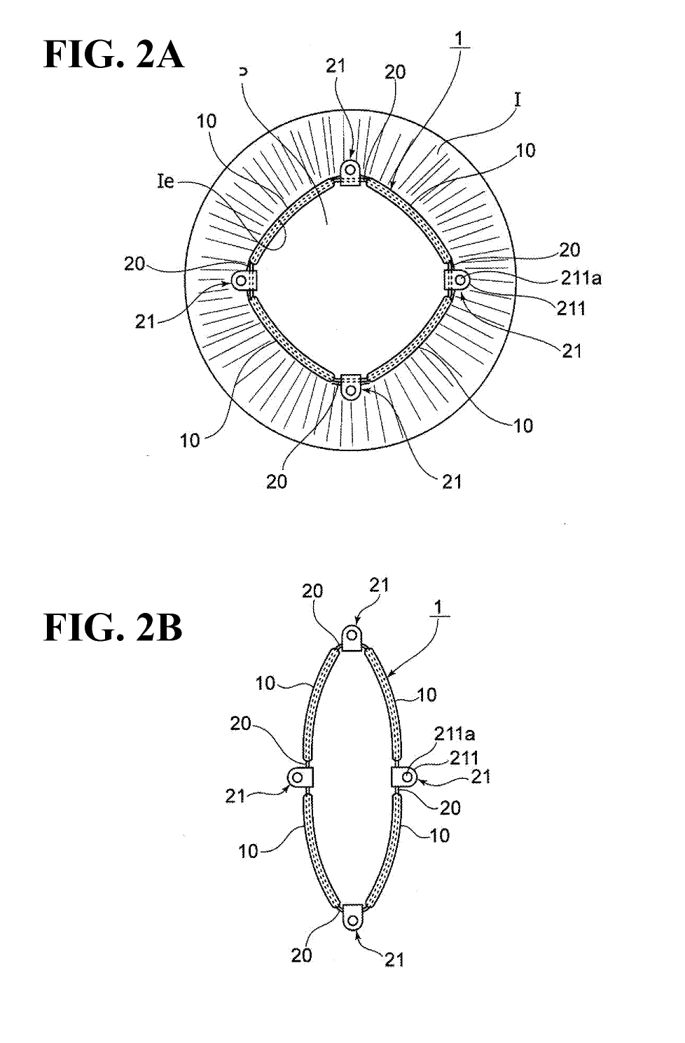

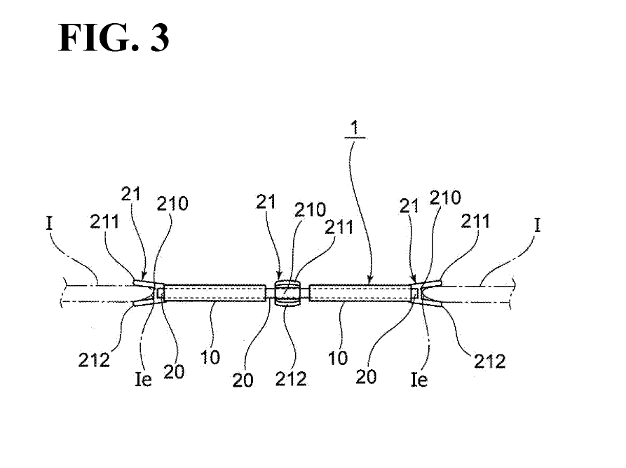

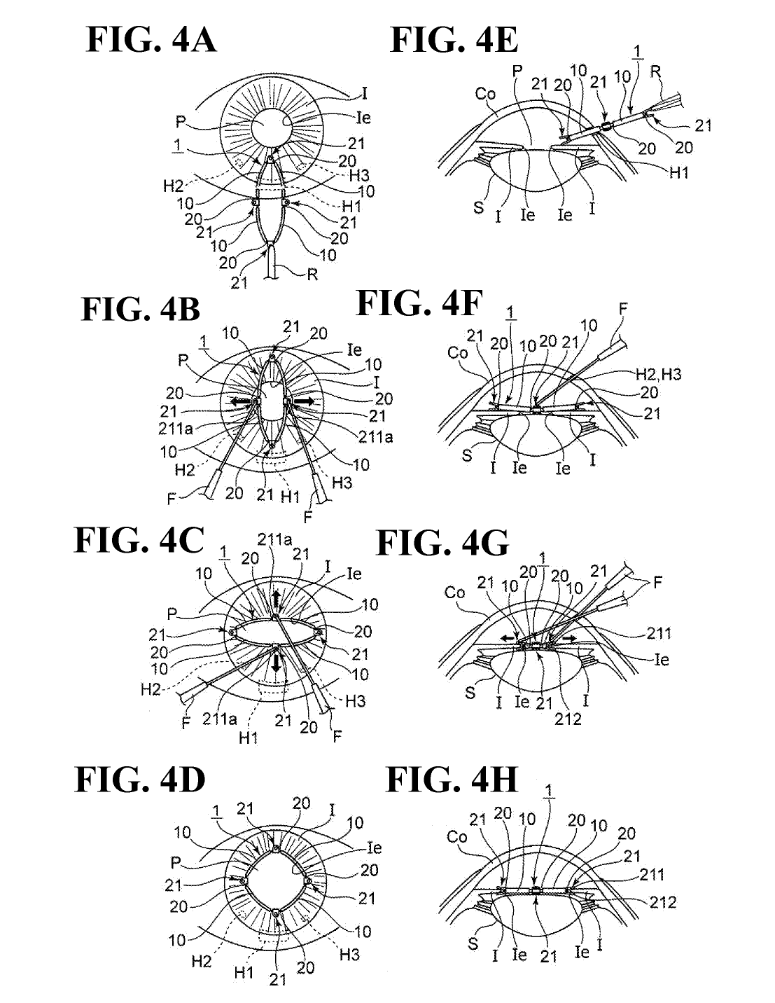

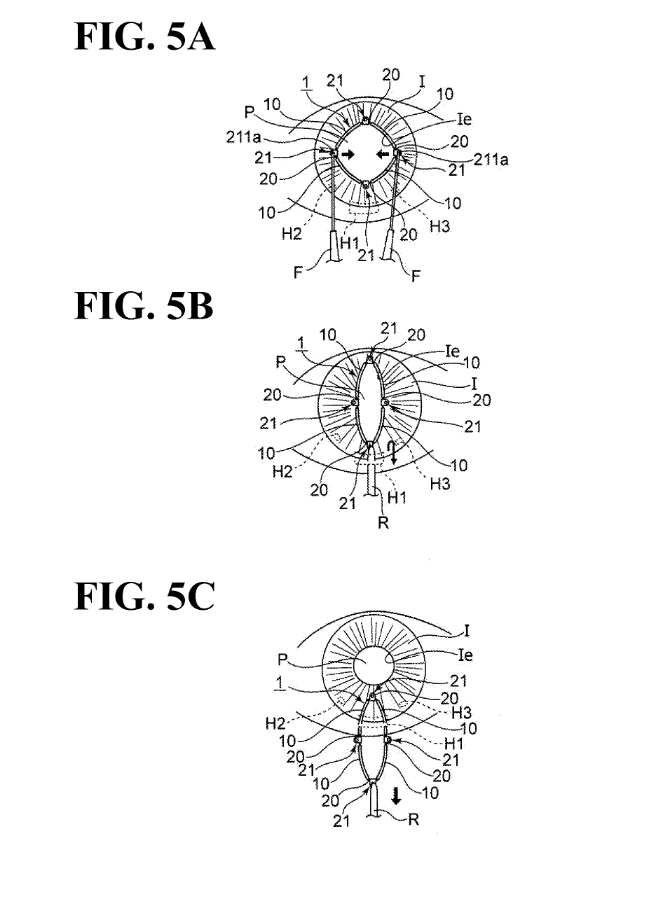

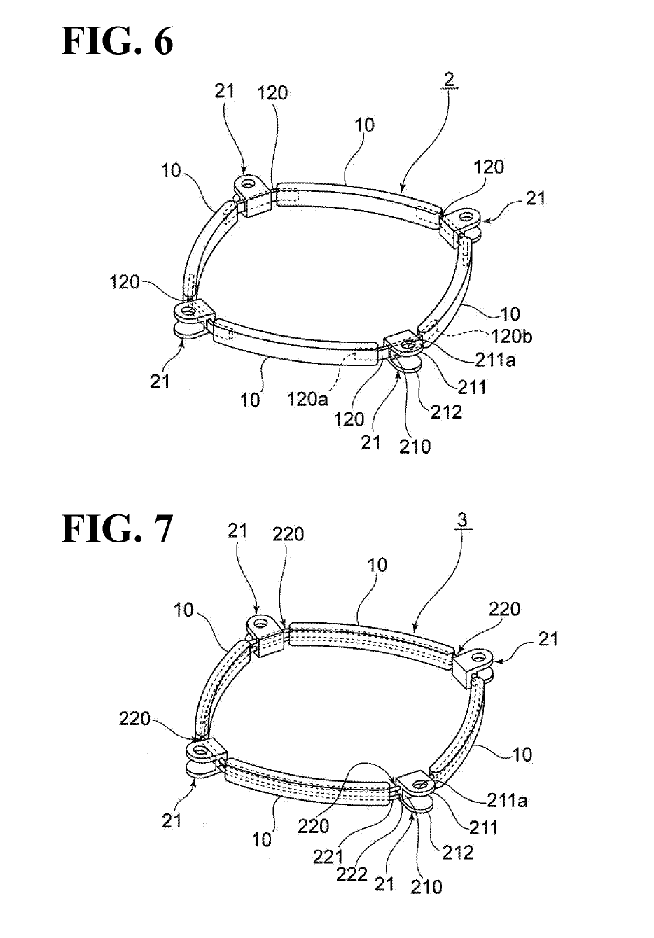

### BRIEF DESCRIPTION OF THE DRAWINGS

[FIG. 1] FIG. 1 is a perspective view of a pupil expander according to a first embodiment.

[FIG. 2] FIG. 2A is a plan view of the pupil expander in an opened state and FIG. 2B is a plan view of the pupil expander in a closed state.

[FIG. 3] FIG. 3 is a side view of the pupil expander in an opened state.

[FIG. 4] FIGS. 4A to 4D are plan views showing arrangement of the pupil expander shown in FIG. 1 in a stepwise manner, and FIGS. 4E to 4H are side views of the arrangement thereof.

[FIG. 5] FIGS. 5A to 5C are plan views showing removal of the pupil expander shown in FIG. 1 in a stepwise manner.

[FIG. 6] FIG. 6 is a perspective view of a modified embodiment according to the pupil expander shown in FIG. 1.

[FIG. 7] FIG. 7 is a perspective view of a pupil expander according to a second embodiment.

### EMBODIMENTS FOR CARRYING OUT THE INVENTION

### First Embodiment

Next, a first embodiment of a pupil expander according to the present invention will be explained with reference to FIGS. 1 to 5.

[Structure of Pupil Expander]

The pupil expander 1 according to this embodiment is used to maintain a pupil P in an expanded state during ophthalmologic surgery such as, e.g., cataract surgery, and, as shown in FIGS. 1 and 2, includes four arm sections 10 arranged in a manner as to form a circular shape, and a coupling section 20 arranged between adjacent arm sections 10.

The arm sections 10 are, as shown in FIGS. 1 and 2, formed in the same size and shape, and each of them is an elongated member extending in a planar direction. Each arm section 10 is formed into a plate shape having a plane extending in an axial direction of the circular shape formed by the arm sections. Therefore, as shown in FIG. 2A, when the pupil expander 1 is arranged at the pupillary edge Ie of the iris I, the radially outward surface of each arm section 10 can engage with the pupillary edge Ie of the iris I.

Further, each arm section 10 is, as shown in FIG. 2A, formed into a slightly curved shape between both end sections thereof, and in cases where arm sections are arranged serially in a manner as to form a circular shape, each arm section is curved in a manner as to expand radially outward. Therefore, according to this, since each arm section 10 is arranged near the iris edge Ie or outside the iris edge Ie of the pupil P, during surgery, the surgical field of the pupil P section can be widened, enabling more easy surgery.

In the arm section 10 of this embodiment, the linear length between both end sections thereof is set to 3.8 mm, the width is set to 0.4 mm, and the thickness is set to 0.3 mm. Further, the arm section 10 is preferably formed by a rigid material such as, e.g., polypropylene, PMMA, or hard silicon.

The coupling section 20 is, as shown in FIGS. 1 and 2, configured to connect end sections of adjacent arm sections 10. In the coupling section 20 of this embodiment, the adjacent coupling sections 20 are connected to thereby form an overall circular shape. Four arm sections 10 are arranged along the circumferential direction of the coupling section 20 with predetermined intervals.

This coupling section 20 is made of a plastic material. In the case of metallic materials, stainless steel, copper, brass, aluminum, silver, etc., can be preferably used. In the case of resin materials, for example, polyolefin, polyethylene, etc., can be preferably used. The plastic material is a material which is easily deformed and has a property that even if an external force added for deformation is removed, distortion remains. With this, the coupling section 20 deforms between end sections of adjacent arm sections 10, which moves the arm sections 10 located on both sides of the coupling section. Even after the deformation, the distortion of the coupling section 20 remains, which keeps the position of the arm sections 10 on both sides. Therefore, at the time of arranging or removing the pupil expander 1, due to the deformation of the coupling section 20, the overall shape of the pupil expander 1 can be deformed, and after the deformation, the overall shape of the pupil expander 1 can be maintained. Therefore, the pupil expander 1 can be arranged safely without damaging the iris I, which enables to maintain the sufficiently expanded state of the pupil P.

Each coupling section 20 is, as shown in FIG. 2, configured to move each arm section 10 in a planar direction with respect to the adjacent arm section 10 within a range of 0 to 180 degrees.

Further, the coupling section 20 is formed into a plate shape having a plane extending in the axial direction of a circle formed by four arm sections 10. With this, the coupling section 20 becomes easily bent in the planar direction of the pupil expander 1, which enables easy movements of each arm section 10 in the planar direction of the pupil expander 1. As a result, the overall shape of the pupil expander 1 can be easily bent in the planar direction. Further, since the coupling section 20 is wide in the axial direction, the coupling section 20 engages with the pupillary edge Ie of the iris I. with a plane. Therefore, when the pupil expander 1 is arranged at the pupillary edge Ie of the iris I as shown in FIG. 2, the expanded state of the pupil P can be simply and assuredly maintained.

The coupling section 20 of this embodiment is set such that the length in the circumferential direction between the end sections of adjacent arm section 10 is 1 mm, the width in the up-down direction is 0.3 mm, and the thickness is 0.1 mm.

Further, the coupling section 20 is, as shown in FIGS. 2 and 3, provided with an iris engagement assisting section 21 for engaging the pupillary edge Ie of the iris I. This iris engagement assisting section 21 is provided with a basal section 210 fixed at the center of the coupling section 20 and engaging ledges 211 and 212 formed into the same size and shape. The engaging ledge 211 is protruded radially outward from the upper end portion of the basal section 210 and the engaging ledge 212 is protruded radially outward from the lower end portion of the basal section 210. Both the engaging ledges face with each other on the outside of the coupling section 20. For this reason, when the pupil expander 1 is arranged at the pupillary edge Ie of the iris I, the pupillary edge Ie of the iris I engages between both the engaging ledges 211 and 212, which enables to safety arrange the pupil expander 1 and also enables to assuredly maintain the expanded state of the pupil P.

Further, in this embodiment, since both the engaging ledges 211 and 212 are formed such that the tip end sections depart gradually as it advances radially outward. This enhances easy insertion of the pupillary edge Ie of the iris I between both the engaging ledges 211 and 212.

Further, in the coupling section 20 is, as shown in FIGS. 1 and 2, a device insertion hole 211a for inserting a medical device for ophthalmologic surgery is formed in the upper engaging ledge 211 of the iris engagement assisting section 21. With this, at the time of arranging and removing the pupil expander 1, by inserting, e.g., a Sinskey hook F into the device insertion hole 211a, it becomes easy to apply a force radially inward or radially outward of the coupling section 20. This enables to easily and assuredly deform the overall shape of the pupil expander 1.

In this embodiment, the iris engagement assisting section 21 is set such that the length in the circumferential direction of the basal section 210 is 0.7 mm. Further, the iris engagement assisting section 21 is preferably made of polypropylene, PMMA, hard silicon, etc. The engagement ledges 211 and 212 are each set such that the length from the basal section 210 is 0.5 mm, the thickness is 0.15 mm, the distance between both engaging ledges 211 and 212 is 0.5 mm, the distance between the tip end sections of both the engaging ledges 211 and 212 is 0.7 mm. Further, the device insertion hole 211a is formed to have a diameter of 0.4 mm.

Considering the scale (length, width, thickness, intervals, etc.) of each structure of the aforementioned pupil expander 1, since the circumferential length of the coupling section 20 is 1 mm, and the circumferential length of the basal section 210 of the iris engagement assisting section 21 is 0.7 mm, the length of each of both the coupling sections 20 outside the iris engagement assisting section 21 is 0.15 mm, and the coupling section 20 is in a deformable state. When the pupil expander 1 is deformed into an elongated substantially rod shape as a whole, the width of the pupil expander 1 can become about 2.2 mm. As a result, it becomes possible to insert the pupil expander into an eye through a very small incision H1 of about 2.5 to 3.0 mm. Further, the length of the pupil expander 1 becomes about 9.8 mm when the width thereof is about 2.2 mm, and therefore when inserting the entire pupil expander 1 into an eye, the risk of damaging the endothelium or the angle of the cornea Co is very low since the inner side long diameter of the cornea Co (the distance from one angle to the other angle) in the eye (in the anterior chamber) is 12 mm. Therefore, when the pupil expander 1 is arranged at the pupillary edge Ie of the iris I, it becomes possible to expand the pupil P so that the maximum diameter becomes 6 mm and the minimum diameter becomes 5.4 mm.

[Method of Arranging and Removing Pupil Expander]

Next, arranging and removing the pupil expander 1 will be explained with reference to FIGS. 4 and 5. Hereinafter, in the cornea Co, preliminarily, an incision H1 for inserting the pupil expander 1 is formed in a manner such that the diameter thereof is about 2.5 to 3.0 mm, and at both right and left sides of the incision H1 in the cornea Co, side ports (incisions) H2 and H3 each for inserting a Sinskey hook F are formed in a manner such that the diameter is about 1.0 mm. These incision H1 and side ports (incisions) H2 and H3 are shown by broken lines in figures.

Initially, when arranging the pupil expander 1, as shown in FIGS. 4A and 4B, a viscoelastic material is injected into the anterior chamber in a manner as to attain the state in which the space between the cornea Co and the iris I is kept deep, and each of the arm sections 10 is moved by deforming the coupling section 20 to thereby deform the pupil expander 1 into a closed state (in an entirely elongated approximately bar shaped state). While pinching one end section (lower end in FIG. 4A) of this pupil expander 1 by a forceps R, the pupil expander 1 is inserted into an eye through the incision H1 so that the pupil expander 1 is arranged above the iris I (see FIG. 4E). As explained above, when the pupil expander 1 is in an elongated approximately bar shape as a whole, the pupil expander 1 is arranged so that the pair of coupling sections 20 (concretely, both engagement ledges 211 and 212 of the iris engagement assisting section 21) facing each other in a proximately approached state are positioned inside the pupillary edge Ie of the iris I.

It is configured such that the pupil expander 1 is naturally arranged at the aforementioned position when the pupil expander 1 is positioned at the center of anterior chamber. Further, when the arrangement position of the pupil expander 1 is inappropriate, the position of the pupil expander 1 may be corrected with two Sinskey hooks F inserted through the side ports H2 and H3. Further, at this stage, in a state in which the lower end section of the pupil expander 1 pinched by the forceps R is protruded from the incision H1, the next operation can be performed. Further, the insertion of the pupil expander 1 into an eye can be performed using an injector.

Next, as shown in FIGS. 4B and 4F, each of the tip end portions of the two Sinskey hooks F inserted through the side ports H2 and H3 is inserted into outside device insertion holes 211a of the pair of coupling sections 20, which is in a closely approached state, the tip end portions are moved in a separating direction (the right-left direction in FIG. 4B) with the pupillary edge Ie of the iris I engaged with the outside portion of the coupling section 20. At this time, the pair of coupling sections 20 in a closely approached state (coupling sections positioned in the right-left direction in FIG. 4A) move both the arm sections 10 so as to decrease the angle between the arm sections 10 positioned on both sides, and the pair of coupling sections 20 in a spaced-apart state (coupling sections positioned in the up-down direction in FIG. 4B) move both the arm sections 10 so as to increase the angle between the arm sections 10 positioned on both sides. When opening the coupling sections 20 in the right-left direction, the coupling sections 20 are opened until the pair of coupling sections 20 in the spaced-apart state (coupling sections positioned in the up-down direction in FIG. 4B) become in a state in which the coupling sections are approached.

Further, when opening the coupling section 20 with the pupillary edge Ie of the iris I engaged between both engaging ledges 211 and 212 of the iris engagement assisting section 21, by positioning the pair of coupling sections 20 on the side of the pupillary edge Ie of the iris I in the previous step, the operation can be easily performed by a cataract surgery operator having an experience of using a Sinskey hook F, etc. Further, since both the upper and lower engaging ledges 211 and 212 are 0.5 mm in length and the same in shape, a compressing force and an expanding force can be applied to the iris I symmetrically in the right-left and front-back directions with a wide area, resulting in less invasion to the iris I.

Next, as shown in FIGS. 4C and 4G, each tip end portion of a Sinskey hook F is inserted into each device insertion hole 211a positioned outside the pair of coupling sections 20 (coupling sections 20 positioned in the up-down direction in FIG. 4C), and the pupil expander 1 is opened in a direction of departing the pair of coupling sections (in the up-down direction in FIG. 4C) with the pupillary edge Ie of the iris I engaged with the outside portion. At this time, the pair of coupling sections 20 in a closely approached state (coupling sections positioned in the up-down direction of FIG. 4C) move both the arm sections 10 so as to decrease the angle between the arm sections 10 positioned on both sides, and the pair of coupling sections 20 in a spaced-apart state (coupling sections positioned in the right-left direction of FIG. 4C) move both the arm sections 10 so as to increase the angle between the arm sections 10 positioned on both sides. When moving the coupling sections 20 in the up-down direction, the coupling sections 20 are moved until the angles between the arm sections 10 become equal.

Thus, as shown in FIGS. 4D and 4H, each arm section 10 and each coupling section 20 engage with the pupillary edge Ie of the iris I. As explained above, the overall shape of the pupil expander 1 is deformed within an eye on the basis of deformations of the coupling sections 20 and the overall shape of the pupil expander 1 is maintained after the deformation. This enables safe arrangement of the pupil expander 1 without damaging the iris I, which in turn can maintain the sufficiently expanded state of the pupil P. Further, since the adjacent coupling sections 20 are connected so that the overall shape is formed into a circular shape, the pupil expander can be safely arranged without damaging the iris I with a simple structure, and it becomes possible to maintain the sufficiently expanded state of the pupil P. After the arrangement of this pupil expander 1, intraocular surgery such as, e.g., cataract surgery is performed in this state.

On the other hand, when removing the pupil expander 1 after completion of the intraocular surgery such as, e.g., cataract surgery, as shown in FIG. 5A, initially, after securing a space in the anterior chamber by injecting a viscoelastic material, the tip end sections of two Sinskey hooks F inserted through the side ports H2 and H3 are inserted into the respective device insertion holes 211a of the pair of coupling sections 20 (coupling sections positioned in the right-left direction in FIG. 5A). Then, the pair of coupling sections 20 are moved in the approaching direction so that the overall shape of the pupil expander 1 is deformed into an elongated substantially rod shape within the eye on the basis of the deformation of the coupling section 20.

Next, as shown in FIG. 5B, one end section of the pupil expander 1 (the lower end section in the figure), which is in an elongated substantially rod shape as a whole, is pinched by forceps R, and as shown in the arrow in the figure, while slightly pushing toward the back side (upward in FIG. 5B) and pulling up, the engaging ledges 211 and 212 of the iris engagement assisting section 21 of one end section of the pupil expander 1 are detached from the pupillary edge Ie of the iris I.

Next, as shown in FIG. 5C, by pulling toward the front side (downward in FIG. 5C) from the state to exenterate through the incision H1 to an outside of the eye. Thus, the pupil expander 1 can be removed.

In this embodiment, the coupling section 20 is formed into a plate shape having a plane extending in the axial direction of the circular shape formed by each arm section 10. However, the coupling section 20 can be formed into another shape.

Further, the iris engagement assisting section 21 is formed such that each of the engaging ledges 211 and 212 is protruded radially outward from the basal section 210 fixed to the coupling section 20, but can be formed into another shape.

Further, the above explanation was directed to the case in which the pupil expander 1 is deformed into an elongated substantially bar shape as a whole at the time of the arrangement. However, the pupil expander 1 can be formed into a preliminarily deformed state. For example, the pupil expander 1 can be preliminarily deformed into an elongated substantially bar shape at the time of shipping it.

### Second Embodiment

Next, a second embodiment of a pupil expander according to the present invention will be explained with reference to FIG. 6. Hereinafter, the explanation will be directed only to the structure different from the aforementioned embodiment, and explanations will be omitted for the same structure by allotting the same symbol.

In the pupil expander 2 of this embodiment is, as shown in FIG. 6, the coupling section 120 is provided between the end sections of adjacent arm sections 10. The one end section 120a of the coupling section 120 is fixed to the end section of the arm section 10 positioned on one side in the circumferential direction, and the other end section 120b of the coupling section 120 is fixed to the end section of the arm section 10 positioned on the other side in the circumferential direction.

### Third Embodiment

Next, a third embodiment of a pupil expander according to the present invention will be explained with reference to FIG. 7 Hereinafter, only the structures different from those of the aforementioned embodiments will be explained, and explanations of the same structures as those of the aforementioned embodiments will be omitted by allotting the same symbol.

In the pupil expander 3 of this embodiment is, as shown in FIG. 7, the coupling section 220 includes two coupling pieces 221 and 222 provided in a manner as to be arranged in parallel in the axial direction of a circular shape (the up-down direction of FIG. 7) formed by arm sections 10, and the coupling pieces 221 and 222 connect the end sections of adjacent arm sections 10. The coupling sections 220 are formed into a circular shape as a whole in a state in which adjacent coupling sections 220 are connected by coupling pieces 221 and 222, and four arm sections 10 are arranged along the circumferential direction of the coupling sections 220 at predetermined intervals.

Since the coupling section 220 is structured as mentioned above, the arm section 10 is supported by two portions in the axial direction, and therefore each arm section 10 can be moved in the planar direction in a stable state.

Further, in this embodiment, the coupling section 220 is formed by two coupling pieces 221 and 222. However, the coupling section 220 can be formed by three or more coupling pieces.

Further, in each of the aforementioned embodiments, the iris engagement assisting section is provided. However, the iris engagement assisting section can be omitted.

Further, the device insertion hole is formed in the iris engagement assisting section. But, the device insertion hole can be formed in the coupling section, or can be omitted.

Further, the arm section is curved in a manner as to expand radially outward. However, the arm section can be formed in a non-curved manner.

Further, the number of the arm sections is four, but can be five or more.

It should be noted that the scale (length, width, thickness, intervals, etc.) of each structure is exemplarily shown as an example, and not limited to it.

Although embodiments of the present invention were explained with reference to the drawings, the present invention is not limited to the illustrated embodiments. Various modifications and/or deformations can be added to the illustrated embodiments within the same scope or within the equivalent range of the present invention.

1. A pupil expander to be used to maintain an expanded state of a pupil during ophthalmologic surgery such as cataract surgery, comprising:
four or more arm sections serially arranged in a manner as to form a circular shape; and
coupling sections each made of a plastic material and configured to connect end sections of the arm sections arranged adjacently.

2. The pupil expander as recited in claim 1, wherein the coupling section is formed into an overall circular shape in a state in which adjacent coupling sections are connected to each other, and the four or more arm sections are arranged at predetermined intervals in a circumferential direction of the coupling sections.

3. The pupil expander as recited in claim 1, wherein the coupling sections are each formed into a plate shape having a plane extending in an axial direction of a circular shape formed by the four or more arm sections.

4. The pupil expander as recited in claim 1, wherein the coupling sections are each formed by a plurality of coupling pieces provided in a manner as to be arranged in parallel to an axial direction of a circular shape formed by the four or more arm sections.

5. The pupil expander as recited in claim 1, wherein the coupling sections are each provided with an iris engagement assisting section for engaging a pupillary edge of an iris.

6. The pupil expander as recited in claim 5, wherein the coupling sections are each provided with a device insertion hole for inserting a medical device for ophthalmologic surgery.

7. The pupil expander as recited in claim 1, wherein the arm section is curved in a manner as to expand radially outward.
